(19) United States
(12) Patent Application Publication
(10) Pub. No.: US 2010/0228037 A1
(43) Pub. Date: Sep. 9, 2010

(54) Process for Preparing Allylmercaptocaptopril (Cpssa) and Related Asymmetrical Disulfides

(75) Inventors: Alexander Khenkin, Rechovot (IL); Aharon Rabinkov, Rechovot (IL); Talia Miron, Kfar-Haim (IL); Meir Wilchek, Rechovot (IL); David Mirelman, Ramat-Efal (IL)

Correspondence Address: MARTIN D. MOYNIHAN d/b/a PRTSI, INC., P.O. BOX 16446, ARLINGTON, VA, 22215, US

(73) Assignee: Yeda Research And Development Co. Ltd. at the Weizmann Institute of Science, Rechovot (IL)

(21) Appl. No.: 12/223,119

(22) PCT Filed: Jan. 24, 2007
(86) PCT No.: PCT/IL2007/000088

§ 371 Date: Jul. 23, 2008

Related U.S. Application Data
(60) Provisional application No. 60/761,770, filed on Jan. 25, 2006.

(51) Int. Cl. C07D 207/16

(52) U.S. Cl. 548/533

(57) ABSTRACT

A novel process of preparing allyl-containing asymmetric disulfides, and particularly therapeutically active allyl-containing asymmetric disulfides such as allylmercaptocaptopril (CPSSA) is disclosed.

### FIELD AND BACKGROUND OF THE INVENTION

The present invention relates to the field of chemical synthesis and more particularly to a novel process of preparing asymmetrical disulfides such as allylmercaptocaptopril (CPSSA).

Captopril (D-3-mercapto-2-methylpropanoyl-L-proline), as well as related azetidine and proline derivatives thereof, are competitive inhibitors of angiotensin-converting enzyme (ACE) which blocks the conversion of angiotensin I to angiotensin II. Thus, captopril and its derivatives have been utilized as therapeutic agents for treating numerous forms of hypertension [see, for example, U.S. Pat. No. 4,046,889, Ondetti et al., Science 196:441-444 (1977); Thind G. S., “Cardiovase Drugs Ther. 4:199-206 (1990); Cushman et al., Hypertension 17:589-592 (1991); Migdalof et al., Drug Metab Rev 15:841-869 (1984); and Materson et al., Arch Intern Med 1544:513-523 (1994)].

Captopril and its derivatives contain an active thiol (—SH) group which binds to the zinc ion in the ACE active site, thus increasing its inhibitory effect.

However, while captopril is substantially stable in aqueous solutions, in the blood or the plasma of mammals (including humans) the active —SH group of captopril easily undergoes oxidation and participates in a thiol-disulfide exchange reaction. This feature accounts for the relatively short duration of ACE inhibition by captopril.

Captopril was further found to bind covalently (although reversibly) to the plasma proteins, via thiol-sulfide exchange reactions with cysteine and glutathione [Migdalof et al. (1984) supra]. This reaction competes with the captopril reaction with the ACE active site and thus reduces the actual amount of an administered captopril that remains available for inhibiting ACE. Hence, relatively large amounts of captopril are typically required to affect ACE inhibition.

Recently, a novel family of captopril conjugates have been disclosed [see, for example, WO 02/096871 and Miron et al., Amer. J. Hypertension, 2004, 17, 71-73, both are incorporated by reference as if fully set forth herein]. A representative member of this family is allylmercaptocaptopril (CPSSA), the product of the reaction between captopril and allicin.

According to the teachings of WO 02/096871 and Miron et al. (supra), CPSSA is prepared via the reaction of allicin and captopril, as depicted in Scheme 1 below.

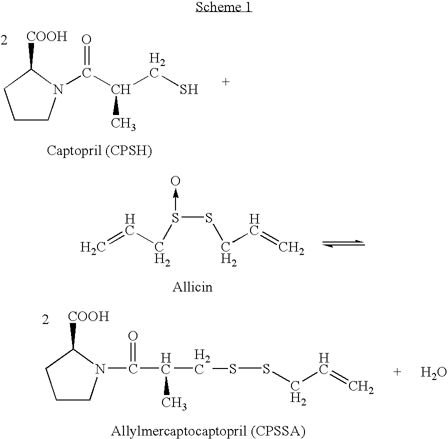

Further according to the teachings of WO 02/096871 and Miron et al. (supra), CPSSA exhibits improved antihypertensive properties, as compared with unmodified captopril.

Allicin, which is used in the preparation of CPSSA, is a biologically active compound derived from garlic. It is naturally produced from the interaction of the enzyme alliinase (alliin lyase; EC 4.4.1.4) with its substrate, alliin (S-allyl-L-cysteine sulfoxide) [A. Stoll and E. Seebeck, Adv. Enzymol. 11 (1951) 377-400].

In the last few years, various studies have demonstrated the many health benefits of allicin, including its effect on hypertension [Elkayam et al., Am. J. of Hypertension 14 (2000) 377-381] and cardiovascular risk factors [Abramovitz et al., Coron. Artery. Dis. 10 (1999) 515-9].

Allicin is an unstable, short-lived molecule that due to its reactivity is able to rapidly react with free thiol groups and penetrate biological membranes with ease [Rabinkov et al. Biochim. Biophys. Acta 1379 (1998) 233-244; Miron et al., Biochim. Biophys. Acta 1463 (2000) 20-30]. Hence, allicin is considered highly potent in affecting different metabolic pathways [K. C. Agarwal, Med. Res. Rev. 16 (1996) 111-124].

However, the high instability of allicin has its drawbacks as allicin disintegrates in the blood a few minutes post its administration both in vitro, in human blood [Freeman and Kodera, J. Agricultural and Food Chem. 43 (1995) 2332-2338], and in vivo, as was demonstrated in rats [Lachmannet al., Arzneimittelforschung 44 (1994) 734-743]. The therapeutic effect of allicin is therefore limited to targets close to the gastrointestinal tract.

While captopril and allicin both are effective agents against hypertonia, each agent operates by a different mechanism.

As discussed above, the use of captopril and allicin alone is limited by high reactivity, instability, and/or competitive reactions.

In contrast, CPSSA and its derivatives, taught in WO 02/096871 and in Miron et al. (supra), combine the advantages of the ACE-inhibiting captopril with the beneficial effects of allicin, while circumventing the limitations associated with each of these components. As further taught in WO 02/096871 and in Miron et al. (supra), CPSSA, as well as derivatives and analogs thereof, react very sluggishly with serum proteins, where the thiol groups are mostly in the disulfide form. Thus, these compounds are stable in blood or plasma of mammals and the high dose requirement so as to achieve an effective anti-hypertensive activity is circumvented. For example, WO 02/096871 shows that CPSSA significantly decreased blood pressure and reduced the serum levels of triglycerides and insulin in rats, to near normal levels, immediately after administration of CPSSA is effected. Similar effects were observed with nearly double doses of captopril per se.

Although CPSSA is obtained by the reaction of captopril and allicin (as depicted in scheme 1 above) in a relatively good yield (about 90%), the process disclosed in WO 02/096871 is limited by the use of allicin as a starting material. As discussed hereinabove, allicin is highly unstable and thus difficult to obtain and handle. In addition, since allicin is the compound responsible for garlic's pungent odor, performing a process that utilizes allicin may involve inconvenience and displeasure.

Asymmetrical disulfides, such as CPSSA, are generally known for their important role in diverse biochemical processes as regulatory hormones, drugs and enzyme activators or inhibitors due to their tendency to disproportionate into symmetrical disulfides under favorable conditions. For example, alkyl 2-imidazolyl disulfide compounds have been shown to act as anti-tumor agents [Hashash et al., J. Pharm. Sci. 91:1686-1692, 2002]. In another example, U.S. Pat. No. 4,049,665 teaches that asymmetrical disulfides of pyridine-1-oxide and acid addition salts thereof were useful as antimicrobial agents. Similarly, U.S. Pat. No. 4,487,780 mentions that the asymmetrical disulfide produced by the reaction of penicillamine and cysteine, is successfully used in the treatment of rheumatoid arthritis.

Several synthetic procedures have been published in the literature for preparing asymmetrical disulfides. These include, for example, the use of starting materials such as diethyl azodicarboxylate [Mukayama et al. Tetrahedron Letters, 1968, 5907], thioimides [Boustany et al. Tetrahedron Letters, 1970, 3547], thionitriles [Street et al J. Chem. Soc. Chem. Commun. 1977, 407], alkylthiosulfates [Swan, Nature, 1957, 180, 143], thioalkoxytrialkylphosphonium salts [Ohmori et al, Chem. Pharm. Bull., 1987, 35, 4473], dithioperoxyesters [Leriverend et al, Synthesis, 1994, 761], alkylthiodialkylsulfonium salt [Dubs et al, Hely. Chim. Acta, 1976, 59, 1307], tosyl thiolates [Field et al, J. Org. Chem. 1968, 33, 3865] and sulfenyl thiocarbonates [Brois et al, J. Amer. Chem. Soc., 1970, 92, 7629].

However, these procedures require the isolation and purification of reaction intermediates such as, for example, an allyl thiosulfate, in order to obtain the product in acceptable yield. Furthermore, performing these procedures in large-scale quantities is very difficult. Even more important, these procedures were developed for sulfides which are soluble in organic solvents, and are not suitable for use with water soluble thiols.

Given the promising therapeutic properties of asymmetrical disulfides in general, and the excellent antihypertensive property of CPSSA in particular, on one hand, and the drawbacks of the known procedures for preparing asymmetrical disulfides, in particular the disadvantages of using allicin, on the other hand, there is a widely recognized need for, and it would be highly advantageous to have, an improved method for the preparation of asymmetrical disulfides in general, and CPSSA in particular, devoid of the above limitations.

### SUMMARY OF THE INVENTION

The present inventors have now designed and successfully practiced a novel process for preparing the promising asymmetric disulfide CPSSA, in which using allicin as a starting material and isolating the intermediate is circumvented and further in which the product is obtained in high yield and purity. This process can be advantageously conducted as a one-pot process and can be readily used for preparing other related asymmetric disulfides.

According to one aspect of the present invention there is provided a process of preparing an allyl-containing asymmetric disulfide, the process comprising reacting an allyl having a reactive group with a thiol-containing compound, in the presence of a thiosulfate, thereby obtaining the allyl-containing asymmetric disulfide.

According to further features in preferred embodiments of the invention described below, the process is a one-pot process.

According to still further features in the described preferred embodiments, the process comprises:

providing a first mixture containing the allyl having the reactive group and the thiosulfate;

subsequently adding a second mixture containing the thiol-containing compound to the first mixture; and

mixing the first mixture and the second mixture.

According to still further features in the described preferred embodiments, providing the first mixture is effected by mixing the allyl having the reactive group and the thiosulfate for a time period that ranges from 1 hour to 20 hours. Preferably, the time period ranges from 10 hours to 20 hours.

According to still further features in the described preferred embodiments, reacting is performed under basic conditions.

According to still further features in the described preferred embodiments, reacting is conducted at a temperature that ranges from about −50° C. to about 50° C.

According to still further features in the described preferred embodiments, reacting is conducted in an aqueous medium.

According to still further features in the described preferred embodiments, a molar ratio between the allyl having a reactive group and the thiol-containing compound ranges from about 10:1 to about 1:10. Preferably, the ratio ranges from about 5:1 to about 1:5.

According to still further features in the described preferred embodiments, a molar ratio between the allyl having a reactive group and the thiosulfate ranges from about 5:1 to about 1:5. Preferably, the ratio is about 1:1.

According to still further features in the described preferred embodiments the reactive group is selected from the group consisting of halide, tosylate and sulfonylchloride.

According to still further features in the described preferred embodiments the reactive group is halide. Preferably, the halide is a bromide.

According to still further features in the described preferred embodiments a concentration of the allyl having a reactive group in the first mixture ranges from about 0.1 M to about 10 M. Preferably, the concentration ranges from about 1 M to about 5 M.

According to still further features in the described preferred embodiments a concentration of the thiosulfate in the first mixture ranges from about 0.1 M to about 10 M. Preferably, the concentration ranges from about 1 M to about 5 M.

According to still further features in the described preferred embodiments the thiol-containing compound is an angiotensin-converting enzyme (ACE)-inhibiting proline derivative.

According to still further features in the described preferred embodiments, the thiol-containing compound is captopril a derivative or an analog thereof.

According to still further features in the described preferred embodiments the thiosulfate is selected from the group consisting of sodium thiosulfate, potassium thiosulfate, calcium thiosulfate, and ammonium thiosulfate.

According to still further features in the described preferred embodiments the process further comprises purifying the allyl-containing asymmetric disulfide.

Preferably, the purifying is effected by column chromatography, recrystallization and/or a combination thereof.

According to still further features in the described preferred embodiments the allyl having a reactive group is allyl bromide, the thiol-containing compounds is captopril and the allyl-containing asymmetric disulfide is CPSSA.

According to another aspect of the present invention there is provided an allyl-containing asymmetric disulfide obtained by the process described herein.

According to yet another aspect of the present invention there is provided a process of preparing an allyl-containing asymmetric disulfide having the general Formula:

A-S—S—B

wherein:

A is a residue of a thiol-containing compound;

B is an allyl residue; and

A and B are different,

the process comprising reacting a compound having the general Formula B—X, wherein X is a reactive group, with a thiol-containing compound having the general Formula A-SH, in the presence of a thiosulfate, thereby obtaining the asymmetric disulfide.

According to further features in preferred embodiments of the invention described below, the process comprises:

providing a first mixture containing the compound having the general Formula B—X and the thiosulfate;

subsequently adding a second mixture containing the compound having the general Formula A-SH to the first mixture; and

mixing the first mixture and the second mixture.

According to still further features in the described preferred embodiments a molar ratio between the compound having the general Formula B—X and the thio sulfate, ranges from about 5:1 to about 1:5. Preferably, the ratio is about 1:1.

According to still further features in the described preferred embodiments, X is halide. Preferably, the halide is bromide.

According to still further features in the described preferred embodiments, a concentration of the compound having the general Formula B—X ranges from 0.1 M to 10M. Preferably, the concentration ranges from about 1 M to about 5 M.

According to still further features in the described preferred embodiments the compound having the general Formula A-SH is an angiotensin-converting enzyme (ACE)-inhibiting proline derivative.

According to still further features in the described preferred embodiments the compound having the general Formula A-SH is captopril, a derivative or an analog thereof.

According to still another aspect of the present invention there is provided an allyl-containing asymmetric disulfide obtained by the process described herein.

According to an additional aspect of the present invention there is provided an allyl-containing asymmetric disulfide having a purity greater than 95%. Preferably, a purity greater than 99%.

According to further features in preferred embodiments of the invention described below, the allyl-containing asymmetric disulfide described herein is allylmercaptocaptopril (CPSSA).

According to still further features in the described preferred embodiments, the B—X is allyl bromide, the A-SH is captopril and the allyl-containing asymmetric disulfide is CPSSA.

The present invention successfully addresses the shortcomings of the presently known configurations by providing an efficient and simple to perform process for preparing asymmetrical disulfides such as CPSSA.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### DESCRIPTION OF THE PREFERRED EMBODIMENTS

The present invention is of a novel process of preparing allyl-containing asymmetrical disulfides and particularly allylmercaptocaptopril (CPSSA), analogs and derivatives thereof. The process utilizes starting materials which are convenient to handle and can be efficiently performed as a one-pot process, while circumventing the need to isolate the intermediates.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As discussed hereinabove, allyl-containing asymmetric disulfide compounds, such as allylmercaptocaptopril (CPSSA) and derivatives and analogs thereof, have been shown to possess excellent antihypertensive properties [WO 02/096871 and Miron et al. (2004) supra]. These highly active allyl-containing asymmetric disulfide compounds (e.g., CPSSA) are conjugates of allicin, a biologically active compound derived from garlic, which has many health benefits, including an effect on hypertension and on cardiovascular risk factors, and of angiotensin-converting enzyme (ACE)-inhibiting proline derivative compounds, such as captopril.

CPSSA, as well as derivatives and analogs thereof, have been shown to be stable in blood or plasma of mammals and thus, their use circumvents the need to use high doses in order to produce an anti-hypertensive effect, which is often the case with non-conjugated ACE-inhibiting proline derivative compounds, such as captopril.

As discussed hereinabove, these novel conjugates, namely CPSSA and its analogs and derivatives, have been prepared by reacting the thiol group of the ACE-inhibiting proline derivative compound with allicin. An exemplary process for preparing CPSSA through the reaction of allicin and captopril is depicted in Scheme 1 hereinabove.

This process, although resulting in a relatively high yield (higher than 90%) is largely affected by the high reactivity of allicin, which is an unstable, short-lived molecule. However, the reactivity of allicin adversely renders this substance difficult to obtain and handle and hence complicates the performance of the above process. In addition, since allicin is the compound responsible for garlic's pungent odor, using it is often very unpleasant and disagreeable.

As discussed hereinabove, allyl-containing asymmetrical disulfides have promising therapeutic properties. However, as further discussed hereinabove, the presently known processes for preparing asymmetrical disulfides in general, and allyl-containing asymmetrical disulfides in particular, suffer many disadvantages, including the inconvenient-to-use starting materials and the laborious, expensive and time-consuming isolation and purification procedures of the intermediates. Some of these procedures are further not suitable for being carried our in aqueous media, with water-soluble thiols.

In a search for a novel and improved process for preparing allyl-containing asymmetrical disulfides, such as CPSSA, the present inventors have envisioned that introducing a thiol-containing allylic species, other than allicin, to a thiol-containing compound, to thereby obtain an allyl-containing asymmetrical disulfide, could serve as an alternative methodology for the preparation of this family of compounds, while circumventing the use of allicin as a starting material. The present inventors have further envisioned that preparing such a species in situ could circumvent the laborious task of separating and purifying the reaction intermediates. It has been further envisioned that such a method could be performed in aqueous media, and would be suitable for practice with water-soluble thiols.

To this end, the present inventors have designed and successfully practiced a novel methodology, which is based on the in situ preparation of an allyl thiosulfate that reacts with a thiol-containing compound, in an aqueous medium, to thereby obtain an allyl-containing asymmetric disulfide.

As is demonstrated in the Examples section that follows, CPSSA, as an exemplary allyl-containing asymmetric disulfide which has promising therapeutic uses, has been successfully prepared according to this methodology, in an exceptionally high purity form. As is further demonstrated in the Examples section that follows, the process was successfully performed as a one-pot process, without isolating the reaction intermediate and was further successfully advantageously performed in an aqueous medium.

Thus, the novel methodology described herein can be advantageously utilized as a novel process for preparing allyl-containing asymmetric disulfides, in which available and easy to handle starting materials are used and the need to isolate the reaction intermediates is circumvented. This process can hence be efficiently scaled-up and utilized in industrial scale while providing highly purified products.

Hence, according to one aspect of the present invention there is provided a process of preparing an allyl-containing asymmetric disulfide. The process is effected by reacting an allyl compound having a reactive group with a thiol-containing compound, in the presence of a thiosulfate.

As used herein, the term “allyl” or “allylic”, also denoted herein as the variable B, describes a chemical species that comprises a —CH₂CH═CH₂ group. This species can form a part of a compound, or may exist as a stable or meta-stable allylic radical or charged species, such as, for example, an allylic cation of the formula ⁺CH₂CH═CH₂. The phrase “allyl-containing” refers to a compound that comprises at least one allyl group. The term “allylic position” refers to the position adjacent to the C═C double bond in the allyl group. For example, an allylic carbon is a carbon atom positioned adjacent to a C═C bond.

The term “disulfide”, as used herein, describes a compound that comprises a disulfide bond (—S—S— bond), also referred to in the art as a disulfide bridge, which is a strong covalent bond between two sulfur radicals. The disulfide bond in a disulfide compound typically links two residues, each being attached to one of the sulfur radicals in the disulfide bond, such that the disulfide compound has the formula R′—S—S—R″.

As used herein, the term “asymmetric disulfide” refers to any compound having a sulfur-sulfur bond which is not a mirror image of itself when split down the sulfur-sulfur bond. This term specifically includes disulfides having the general formula R′—S—S—R″ as well as (bis)disulfides having the general formula of R′—S—S—Y—S—S—R″, wherein R′, R″ and Y (if present) are any residue or group that can be attached to the sulfur radical in the disulfide bond(s), including, for example, alkyl, cycloalkyl, allyl, alkenyl, alkynyl, aryl, heteroaryl, heteroalicyclic, amine, hydroxy, and more, provided that R′ and R″ are different residues or groups.

Thus, by the term “asymmetric disulfide” it is meant that the groups on either side of a disulfide bond are different, such that R′ and R″ in the formula above differ from one another.

Furthermore, the term “asymmetric disulfide” as used herein also encompasses all biochemical equivalents of the particular asymmetric disulfide being referenced, namely, salts, prodrugs, precursors and the like.

The phrase “allyl-containing asymmetric disulfide” therefore describes an asymmetric disulfide, as defined herein, which contains at least one allyl group, as defined herein.

An allyl-containing asymmetric disulfide is further represented herein by the general Formula I:

A-S—S—B Formula I

wherein A is a residue of a thiol-containing compound, as this term is defined herein; and B is an allyl residue or an allyl-containing residue, as this term is defined herein, whereby A and B are not the same.

Preferably, the allyl-containing asymmetric disulfides obtained by the process of the present embodiments are allylic derivatives of ACE-inhibiting proline compounds.

According to the presently most preferred embodiment of the present invention, the allyl-containing asymmetric disulfides include allylmercaptocaptopril (CPSSA) analogs, salts and chemical derivatives thereof.

As used herein throughout, the term “analogs” refers to compounds that are structurally related to the subject molecule (e.g., CPSSA) and can therefore exert the same biological activity.

The term “derivatives” refers to subject molecules (e.g., CPSSA) which has been chemically modified but retain a major portion thereof unchanged. Non-limiting examples include subject molecules which are substituted by additional or different substituents, subject molecules in which a portion thereof has been oxidized or hydrolysed, and the like.

The term “reactive group” which is further denoted herein as X, is used herein in the context of an “an allyl having a reactive group”. This term, as used herein, describes a chemical group that is capable of undergoing a chemical reaction that typically leads to a bond formation. The bond can be a covalent bond, an ionic bond, a hydrogen bond and the like and is preferably a covalent bond. Chemical reactions that lead to a bond formation include, for example, nucleophilic and electrophilic substitutions, nucleophilic and electrophilic addition reactions, elimination reactions, cyclo-addition reactions, rearrangement reactions, aromatic interactions, hydrophobic interactions, electrostatic interactions and any other known reactions that result in an interaction between two or more components.

Since the nature of the reactions involved in the process described herein are mainly nucleophilic, exemplary reactive groups that are suitable for use in the context of the present invention are leaving groups.

As used herein, the phrase “leaving group” describes a labile atom, group or chemical moiety that readily undergoes detachment from an organic molecule during a chemical reaction, while the detachment is facilitated by the relative stability of the leaving atom, group or moiety thereafter. Typically, any group that is the conjugate base of a strong acid can act as a good leaving group. Representative examples of suitable leaving groups according to the present embodiments therefore include, without limitation, acetate, tosylate, hydroxy, thiohydroxy, alkoxy, halide, sulfonylhalide, amine, azide, cyanate, thiocyanate, nitro and cyano.

Preferably, the leaving group is halide.

The term “halide” or “halo” describes fluoride, chloride, bromide or iodide.

More preferably, the halide is a bromide.

As can be seen in the Examples section which follows, conducting the reaction with allyl bromide gave better results (higher yield and lower percentage of unreacted captopril) compared to the same reaction with allyl chloride (see Examples 1-4 with allyl bromide, versus Examples 5-6 with allyl chloride).

The term “thiosulfate”, as used herein, describes a compound which contains a thiosulfate group. A “thiosulfate group” describes a S₂O₃⁻² group, which can be represented by the formula —(S—S(═O)₂—O)⁻². The thiosulfate can therefore include a thiosulfate group, which is an anion, and a cation, thus being a thiosulfate salt. Examples of thiosulfate salts include, but are not limited to, sodium thiosulfate, potassium thiosulfate, calcium thiosulfate, and ammonium thiosulfate. The thiosulfate salt may be either anhydrous or in the form of a hydrate thereof.

The term “hydrate” refers to a complex of variable stoichiometry (e.g., di-, tri-, tetra-, penta-, hexa-, and so on), which is formed between a compound (e.g., a thiosulfate) and water. Typically, the water molecules are bound to the compounds by non-covalent intermolecular forces.

As used herein, the term “thiol”, which is also known in the art and referred to herein interchangeably as “thiohydroxy”, describes a —SH group.

The phrase “thiol-containing compound”, which is also denoted herein as A-SH, refers to a compound which contains at least one —SH group. In this respect, A is defined as a residue of said thiol-containing compound.

Since according to the presently most preferred embodiments of the invention, the resulting asymmetrical disulfide prepared by the process described herein is CPSSA, preferred thiol-containing compounds according to the present embodiments include captopril (CPSH), derivatives and analogs thereof, as well as other thiol-containing ACE-inhibiting proline derivatives.

It should be noted that in cases where the thiol-containing compound includes one or more asymmetric functions, all stereoisomers (e.g., enantiomers, diastereomers) and racemic forms thereof are encompassed by the present embodiments.

The novel process presented herein is therefore based on a novel methodology in which the allyl-containing asymmetric disulfide is obtained through an in situ preparation of an allyl thiosulfate from an allyl compound and a thiosulfate, and a subsequent reaction of the in situ obtained allyl thiosulfate with a thiol-containing compound.

This methodology can serve to prepare allyl-containing asymmetric disulfides which are actually conjugates of a thiol-containing compound and a desired allyl residue, covalently linked via a disulfide bond. As discussed hereinabove, an exemplary such conjugate is CPSSA, in which a residue of allicin is linked to a residue captopril. The beneficial therapeutic effects of CPSSA are attributed to the presence of these two residues in the compounds.

Thus, by appropriately selecting the allyl compound and the thiosulfate that compose the in situ prepared allyl thiosulfate, and the thiol-containing compound, any desired allyl-containing asymmetric disulfide conjugate can be prepared using this process.

As detailed in the Examples section which follows, CPSSA was prepared according to this methodology, by the in situ preparation of allyl thiosulfate from allyl bromide and sodium thiosulfate, and the subsequent reaction of the in situ obtained allyl thiosulfate with captopril.

The allyl thiosulfate used in the preparation of CPSSA, according to the present embodiments, is selected such that in the final product the allyl residue that is linked to the captopril residue is the same as the residue obtained by reacting captopril and allicin. Thus, CPSSA can be synthetically prepared by this methodology while circumventing the use of allicin itself, which is both difficult to obtain and handle and has an unpleasant and disagreeable odor.

As is further discussed hereinabove, the presently known methods of preparing asymmetric disulfides in general, and allyl-containing asymmetric disulfides in particular, suffer the disadvantage of requiring the separation, isolation or purification the intermediate products obtained in the process. These intermediate products include, for example, allylthiosulfates.

The present inventors have now surprisingly uncovered that by using the methodology described herein, allyl-containing asymmetric disulfides can be conveniently prepared in a one-pot process, while circumventing the need to isolate and purify the intermediate product(s), whereby the allyl-containing asymmetric disulfides are obtained in a remarkably high purity, as is detailed hereinbelow.

Performing the process as a one-pot process is highly advantageous since, by circumventing the need to isolate and purify the intermediates, the process is simplified and is cost-effective and thus can be easily scaled-up.

The phrase “one-pot process”, as used herein, describes a process in which all the reactions and/or procedures involved in the process are performed, either simultaneously or sequentially, without the necessity of any separation, isolation or purification procedures of any intermediate products. The various reactions and/or procedures can be conducted either simultaneously, consequently or at intervals. To clear any doubt, it should be noted that this phrase does not necessarily refers to a process that is literally performed in a single pot. Hence, a process which includes removal of certain mechanical impurities by, for example, physical means such as filtration or decantation is also encompassed by this phrase.

According to preferred embodiments of the present invention, the process described herein is effected by providing a first mixture which contains an allyl having a reactive group, as defined herein, and a thiosulfate salt. To the first mixture, a second mixture containing a thiol-containing compound is added and the two mixtures are mixed to thereby obtain the required product.

Further according to preferred embodiments of the present invention, the first mixture is obtained by simply mixing (by, e.g., stirring) the allyl having a reactive group and the thiosulfate salt. Without being bound to any particular mechanism, it is assumed that the allylic compound and the thiosulfate form, in situ, the reactive species allyl thiosulfate, which thereafter react with the added thiol-containing compound, so as to provide the final desired product.

Further according to preferred embodiments of the present invention, the second mixture is obtained by simply dissolving the thiol-containing compound in an aqueous solvent.

In a search for the optimal conditions for performing the process described herein, the present inventors have performed the process repeatedly, while testing the effect of various parameters of the process efficiency in terms of the yield and purity of the final product.

As is demonstrated in the Examples section that follows, a few parameters have been found to affect the process efficiency. These include the time length of the reaction, the pH conditions, the concentrations of the allyl having a reactive group and of the thiosulfate, and the molar ratio between the allyl having a reactive group and the thiol-containing compound.

Thus, for example, it has been found that mixing of the allyl having said reactive group and the thiosulfate, should preferably be effected for a time period that ranges from about 1 hour to about 20 hours. As can be seen in the Examples section which follows (see, for example, Examples 1-6), satisfactory results have been obtained when mixing the allyl having said reactive group and the thiosulfate was effected during 3, 5, 10 (overnight) and 15 hours, whereby the best results were obtained when this mixing was performed during 10 (overnight) and 15 hours.

As used hereinafter the term “about” refers to ±10%.

Thus, mixing the allyl having said reactive group and the thiosulfate to obtain the first mixture is preferably effected during a time period that ranges from about 1 hour to about 20 hours, more preferably from about 5 hours to about 20 hours and more preferably from about 10 hours to about 20 hours

It has been further found that the reaction should preferably be performed under basic conditions. By the term “basic conditions” it is referred to a pH of the reaction mixture which is higher than 7. Maintaining such a pH is preferably achieved by performing the process in the presence of a buffer. Suitable buffers for use in this context of the present invention include, for example, phosphate buffers such as a disodium hydrogen orthophosphate buffer.

Thus, according to preferred embodiments of the present invention, the process is performed under basic conditions. According to further embodiments of the present invention, the process is performed in the presence of a buffer, as described herein.

It should be noted that the concentrations appearing throughout the specification, Examples and claims, all relate to the mother solutions, namely, the concentration of each reactant in a solution before mixing it in a reaction solution. Thus, the allyl and thiosulfate concentrations as referred to hereinbelow relate to the concentrations of these reactants in the first mixture. Similarly, the concentration of the thiol-containing compound referred to hereinbelow relates to the concentration of this reactant in the second mixture (i.e. before it is mixed with the other reactants).

Considering the above, it has been shown that the process can be effected using solutions in which the concentration of both the allyl having a reactive group, and of the thiosulfate, each preferably ranges from about 0.1 M to about 10 M. More preferably, the concentration of each of these reactants ranges from about 1 M to about 5 M.

As can be seen in Scheme 2 below, which depicts the preparation of CPSSA according to the methodology of the present invention, the stoichiometric ratio of the allyl having a reactive group and the thiol-containing compound is a 1:1 molar ratio. However, while reducing the present invention to practice, it has been found that the reaction can be preferably effected at a molar ratio that ranges from about 10:1 to about 1:10, more preferably, from about 5:1 to about 1:5. It has particularly been found that even more preferably, the allyl having a reactive group is kept in excess compared to the thiol-containing compound.

As is demonstrated in the Examples section that follows, conducting the reaction in a large excess of the allylic reagent, resulted in exceptionally high purities (greater than 99%) and relatively high yields (greater than 60%) of the obtained CPSSA product. For example, a molar ratio of 4:1 allyl:CPSH resulted in a product characterized by a 99.5% purity and obtained in 71% yield (see, Example 1); a molar ratio of 2.6:1 allyl:CPSH resulted in a product characterized by a 98.7% purity and obtained in 60% yield (see, Example 1), and a molar ratio of 1.4:1 allyl:CPSH resulted in a product characterized by a 96% purity and obtained in 59% yield (see, Example 2).

The process can be effected at a temperature that ranges from about −50° C. to about 50° C. Preferably, mixing the first and second mixtures is conducted at ambient temperatures, between −10° C. to +30° C. In a preferred embodiment of the present invention, the mixing is conducted at ambient temperature (e.g., room temperature (rt), 15 to 30° C.), and is followed by a cooling stage, preferably to about 0° C., in order to facilitate the precipitation of the product.

The process can further be effected in the presence of a solvent. While any solvent can be used, given the solubility of the reagents in water, it is desirable to conduct the reaction in an aqueous medium. Thus, in a preferred embodiment of the present invention, the process is effected in an aqueous solvent, which is preferably water.

Performing the process in an aqueous media is safe, cost-effective, environmentally-friendly and hence highly suitable for a scaled-up process. As discussed in detail in the Background section, the currently known processes for the preparation of asymmetric disulfides, by being focused on reactions in organic media, are not suitable for use with water soluble reagents, such as thiols.

Further according to preferred embodiments of the present invention, the molar ratio of reacting allyl having a reactive group and the thiosulfate can range from about 5:1 to about 1:5. Preferably, this ratio is a stoichiometric 1:1 molar ratio.

The preparation of the allyl-containing asymmetric disulfides may be followed by purifying the obtained product. Thus, in a preferred embodiment of the present invention the product obtained in the process described hereinabove is subjected to one or more purification procedures. Preferably, purifying the allyl-containing asymmetric disulfide is effected by one or more of extraction, column chromatography, and recrystallization.

As detailed in the Examples section which follows, the allyl-containing asymmetric disulfides obtained by the process are characterized by a high degree of purity. As is demonstrated in Examples 1-4 below, by following the methodology of the present invention, compounds having a purity of 95% and higher (96% and 97%) can be obtained. In fact, purities over 99% have been easily obtained using the above methodology.

Thus, according to yet another aspect of the present invention, there is provided an allyl-containing asymmetric disulfide having a purity greater than 95%. Preferably, the allyl-containing asymmetric disulfide has a purity greater than 97% and more preferably greater than 99%.

Such an exceptionally high purity is most advantageous in pharmacological and analytical uses, where it is necessary to use reagents of a high purity level.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non limiting fashion.

### Materials and Analytical Methods

Captopril was obtained from Teva, Israel. All other chemicals were obtained from Sigma, Aldrich and Merck.

Product separation was conducted on HPLC using a LiChrosorb RP-18 (7 mm) column and a mixture of 60% methanol in water containing 0.05% trifluoroacetic acid as a mobile phase. Flow rate was kept at 1.0 ml/minute.

Product identification and separation were conducted by HPLC using a LiChrosphere 100 RP-18 (5 mm) column and a mixture of 60% methanol in water containing 0.05% trifluoroacetic acid as a mobile phase; flow rate was kept at 1.0 ml/minutes; and the detector was operated at 220 nm. Retention times were 3.1 minutes for captopril and 9.7 minutes for CPSSA.

The purity of the products was verified using ¹H-NMR, IR, UV-visible and melting point measurements.

¹H-NMR spectra were recorded on a Bruker Avance 250 DPX instrument using CDCl₃ solutions; the chemical shifts are expressed in ppm, downfield from Me₄Si as internal standard.

IR spectra were recorded on Nicolet Protégé 460 FTIR instrument, using KBr pellets.

UV spectra were recorded on a HP 8452A diode array UV-vis spectrometer, for solutions of 0.15 mg CPSSA in 2 ml of a 1:1 MeOH:H₂O mixture.

Melting point was determined using a Stuart Scientific SMP 10 instrument with a temperature gradient of 2° C./minute.

### Chemical Syntheses

The general process for preparing allylmercaptocaptopril (CPSSA) according to the preferred embodiments of the present invention is illustrated in Scheme 2 below. This process was performed while testing the effect of various parameters on its efficiency, as demonstrated in Examples 1-6 that follow.

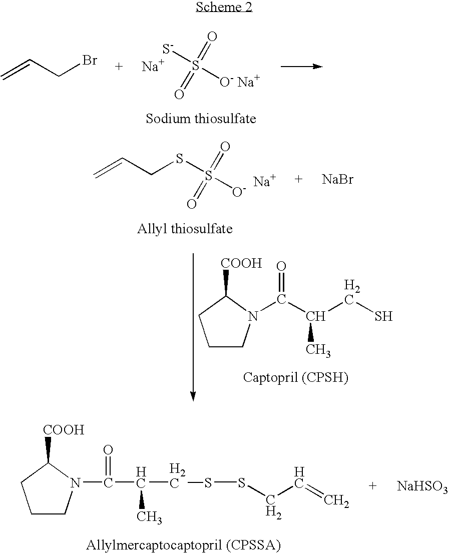

### Example 1

Allyl bromide (48 ml, 0.556 mol) was added to a solution of anhydrous sodium thiosulfate (88.2 grams, 0.558 mol) in water (189.6 ml), and about one minute thereafter the solution turned yellow. The resulting mixture was then stirred overnight at room temperature and was kept in the cold room (at about 4° C.) for storage. The solution was filtered before usage to remove mechanical impurities. Then, part of this solution (197 ml, 0.4 mol) was transferred to another flask and was mixed with a 0.5 M disodium hydrogen orthophosphate solution (34 ml).

In a separate flask, a homogeneous captopril (CPSH) solution was prepared by dissolving captopril (21.7 grams, 0.1 mol) in a 0.5 M disodium hydrogen orthophosphate solution (170 ml) while heating the mixture in a water bath under nitrogen atmosphere. The captopril solution was added to the flask containing the previously prepared allyl thiosulfate solution and stirring was continued under nitrogen atmosphere for 2 hours at room temperature, followed by cooling in an ice bath (to about 5° C.). 4M HCl (15.9 ml) was added to the cooled mixture, so as to acidify the mixture to a final pH of about 3, and a precipitation of a white solid was observed. The mixture was then placed in a cold room (at about 4° C.) for 5 days, and was monitored on a daily basis by HPLC. After 5 days, the precipitate was washed with water, and the crude product was twice slurried in water and filtered. The precipitate was thereafter dried in the air, followed by additional drying in a dessicator over P₂O₅ under vacuum. The product was then slurried in hexane, and filtered. After drying in a dessicator, pure allyl mercaptocaptopril (CPSSA) was obtained (20.87 grams, 0.086 mol, 72% yield).

The purity of the final product, as determined by HPLC, was 99.46%.

¹H-NMR (CDCl₃): δ=1.18 (d, 3H), 2.20 (m, 4H), 2.64 (m, 1H), 3.03 (m, 2H), 3.29 (d, 2H), 3.64 (t, 2H), 4.55 (m, 1H), 5.15 (m, 2H, allyl), 5.80 (m, 1H, allyl) ppm.

melting point: 44° C.

IR: ν=2968, 1604, 1329, 1187, 924 cm⁻¹.

UV-VIS: λ max=211 nm

The same experiment was repeated using 128 ml (0.26 mol) of the allyl thiosulfate solution, also obtaining a high purity product (98.74% purity, 17.33 grams, 0.071 mol, 60% yield).

### Example 2

Allyl bromide (17.3 ml, 0.2 mol) was added to a solution of sodium thiosulfate pentahydrate (49.6 grams, 0.2 mol) in water (50 ml) and the resulting mixture was stirred at room temperature for 15 hours until the initial two phases disappeared and the reaction mixture became homogeneous. Disodium hydrogen orthophosphate (50 ml of a 0.5 M aqueous solution having a pH of about 8.0) was added and the resulting allyl thiosulfate solution was cooled in an ice bath. A captopril (CPSH) solution was prepared by dissolving captopril (32 grams, 0.147 mol) in a 0.5 M disodium hydrogen orthophosphate solution (250 ml) under nitrogen atmosphere. The captopril solution was added, under stirring and bubbling of nitrogen, to the allyl thiosulfate solution, while maintaining the pH of the reaction mixture at about 8.0. Stirring was continued for 1 hour at 0° C. and for another hour at room temperature. The reaction mixture was thereafter cooled again in an ice bath, and 4M HCl (35 ml) was added, under nitrogen atmosphere, so as to acidify the mixture to a final pH in the range of 2-3. The mixture was then placed in a refrigerator (+4° C.) for 4 days, until precipitation of a white solid was observed. The precipitate was filtered, carefully washed with water, and dried under vacuum. The crude product (23 grams) was treated with n-hexane (100 ml) for 1 hour and the white solid was thereafter filtered and dried in vacuum to give pure CPSSA (21 grams, 0.086 mol, 59% yield).

The purity of the final product, as determined by HPLC, was 96%.

¹H NMR (CDCl₃): δ=1.18 (d, 3H), 2.20 (m, 4H), 2.64 (m, 1H), 3.03 (m, 2H), 3.29 (d, 2H), 3.64 (t, 2H), 4.55 (m, 1H), 5.15 (m, 2H allyl), 5.80 (m, 1H allyl) ppm.

melting point: 44° C.

IR: ν=2968, 1604, 1329, 1187, 924 cm⁻¹.

UV-vis λₘₐₓ=211 nm.

### Example 3

Allyl bromide (17.3 ml, 0.2 mol) was added to a solution of sodium thiosulfate pentahydrate (49.6 grams, 0.2 mol) in water (50 ml), and the mixture was stirred at room temperature for 5 hours until the initial two phases disappeared and the reaction mixture became homogeneous. The reaction mixture was then cooled and stirred in an ice bath. A captopril (CPSH) solution was prepared by dissolving captopril (32 grams, 0.147 mol) in a 0.5 M disodium hydrogen orthophosphate solution (300 ml) under nitrogen atmosphere. The captopril solution was added to the reaction mixture, under stirring and bubbling of nitrogen, while maintaining the pH of the reaction mixture at about 8.0 and monitoring the reaction progress by HPLC. Stirring was continued for 30 minutes at 0° C. and the temperature was then raised to room temperature. 4M HCl (35 ml) was added, under nitrogen atmosphere, so as to acidify the mixture to a final pH in the range of 2-3. The mixture was thereafter extracted with ethyl acetate (3 aliquots of 150 ml each) and the organic phase was dried over Na₂SO₄, filtered, evaporated and dried under vacuum, to afford the crude product as an oil (26 grams) containing 75% of CPSSA and 22% of captopril, as determined by HPLC. The CPSSA was purified by column chromatography as follows: Crude CPSSA (5 grams) was dissolved in ethyl acetate and the solution loaded onto a column (10×100 cm) packed with Silica gel 60 pre-equilibrated with hexane. The column was first eluted with 600 ml of a 60:40 ethyl acetate:hexane mixture, and then with a 5:35:60 methanol:ethyl acetate:hexane mixture, to give semi-pure CPSSA as a solid (15 grams, 0.052 mol, 36% yield) having a purity of 92%, as determined by HPLC. Re-crystallization of the semi-pure solid from a diethylether-hexane mixture (1:1) gave 11 grams (0.045 mol, 31% yield) of the highly pure CPSSA.

The purity of the final product, determined by HPLC, was 97%.

¹H-NMR (CDCl₃): δ=1.18 (d, 3H), 2.20 (m, 4H), 2.64 (m, 1H), 3.03 (m, 2H), 3.29 (d, 2H), 3.64 (t, 2H), 4.55 (m, 1H), 5.15 (m, 2H allyl), 5.80 (m, 1H allyl) ppm.

Melting Point: 44° C.;

IR: ν=2968, 1739, 1604, 1326, 1178 cm⁻¹

### Example 4

Allyl bromide (0.173 ml, 2 mmol) was added to a solution of sodium thiosulfate pentahydrate (500 mg, 2 mmol) in water (5 ml), and the resulting mixture was stirred at room temperature for 15 hours until the initial two phases disappeared and the reaction mixture became homogeneous. The reaction mixture was then cooled and stirred in an ice bath. A captopril (CPSH) solution was prepared by dissolving captopril (432 mg, 2 mmol) in 3 ml of a 0.5 M disodium hydrogen orthophosphate solution under nitrogen atmosphere. The captopril solution was added, under stirring and bubbling of nitrogen, to the allyl thiosulfate mixture, while maintaining the mixture pH at about 8.0. Stirring was continued while monitoring the reaction progress by HPLC. After 30 minutes of stirring at 0° C. the reaction mixture contained 70% of CPSSA and 15% of unreacted captopril, as determined by HPLC.

### Example 5

Allyl chloride (0.144 ml, 2 mmol) was added to sodium thiosulfate pentahydrate (500 mg, 2 mmol) in 5 ml of water, and the mixture was stirred at room temperature for 3 hours until the initial two phases disappeared and the reaction mixture became homogeneous. The reaction mixture was then cooled and stirred in an ice bath. A captopril (CPSH) solution was prepared by dissolving captopril (432 mg, 2 mmol) in a 0.5 M disodium hydrogen orthophosphate solution (3 ml) under nitrogen atmosphere. The captopril solution was added, under stirring and bubbling of nitrogen, to the reaction mixture, while maintaining the pH of the reaction mixture at about 8.0. Stirring was continues while monitoring the reaction progress by HPLC. After 40 minutes of stirring at 0° C., the reaction mixture contained 30% of CPSSA and 20% unreacted captopril, as well as other products, as determined by HPLC.

### Example 6

Allyl chloride (0.720 ml, 10 mmol) was added to a solution of sodium thiosulfate pentahydrate (2.48 grams, 10 mmol) in water (15 ml), and the resulting mixture was stirred at room temperature for 15 hours until the initial two phases disappeared and the reaction mixture became homogeneous. The water was thereafter evaporated in vacuum and the precipitated white solid (1.76 grams) was washed with ethyl alcohol, dried and re-dissolved in a 0.5 M disodium hydrogen orthophosphate buffer (10 ml). The mixture was cooled and stirred in ice bath. A captopril (CPSH) solution was prepared by dissolving captopril (920 mg, 4.2 mmol) in a 0.5 M disodium hydrogen orthophosphate solution (3 ml) under nitrogen atmosphere. The captopril solution was added, under stirring and bubbling of nitrogen, to the allyl thiosulfate mixture, while maintaining the pH of the reaction mixture at about 8.0. Stirring was continues while monitoring the reaction progress by HPLC. After 30 minutes of stirring at 0° C., the reaction mixture contained 38% of CPSSA and 43% unreacted captopril, as determined by HPLC.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A process of preparing an allyl-containing asymmetric disulfide, the process comprising reacting an allyl having a reactive group with a thiol-containing compound, in the presence of a thiosulfate, thereby obtaining the allyl-containing asymmetric disulfide.

2. The process of claim 1, being a one-pot process.

3. The process of claim 1, comprising:
providing a first mixture containing said allyl having said reactive group and said thiosulfate;
subsequently adding a second mixture containing said thiol-containing compound to said first mixture; and
mixing said first mixture and said second mixture.

4. The process of claim 1, wherein providing said first mixture is effected by mixing said allyl having said reactive group and said thiosulfate for a time period that ranges from 1 hour to 20 hours.

5. The process of claim 4, wherein said time period ranges from 10 hours to 20 hours.

6. The process of claim 1, wherein said reacting is performed under basic conditions.

7. The process of claim 1, wherein said reacting is conducted at a temperature that ranges from about −50° C. to about 50° C.

8. The process of claim 1, wherein said reacting is conducted in aqueous medium.

9. The process of claim 1, wherein a molar ratio between said allyl having a reactive group and said thiol-containing compound ranges from about 10:1 to about 1:10.

10. The process of claim 9, wherein said ratio ranges from about 5:1 to about 1:5.

11. The process of claim 1, wherein a molar ratio between said allyl having a reactive group and said thiosulfate, ranges from about 5:1 to about 1:5.

12. The process of claim 11, wherein said ratio is about 1:1.

13. The process of claim 1, wherein said reactive group is selected from the group consisting of halide, tosylate and sulfonylchloride.

14. The process of claim 1, wherein said reactive group is halide.

15. The process of claim 14, wherein said halide is a bromide.

16. The process of claim 1, wherein a concentration of said allyl having a reactive group in said first mixture ranges from about 0.1 M to about 10 M.

17. The process of claim 16, wherein said concentration ranges from about 1 M to about 5 M.

18. The process of claim 1, wherein a concentration of said thiosulfate in said first mixture ranges from about 0.1 M to about 10 M.

19. The process of claim 18, wherein said concentration ranges from about 1 M to about 5 M.

20. The process of claim 1, wherein said thiol-containing compound is an angiotensin-converting enzyme (ACE)-inhibiting proline derivative.

21. The process of claim 1, wherein said thiol-containing compound is captopril, a derivative or an analog thereof.

22. The process of claim 1, wherein said thiosulfate is selected from the group consisting of sodium thiosulfate, potassium thiosulfate, calcium thiosulfate, and ammonium thiosulfate.

23. The process of claim 1, further comprising purifying the allyl-containing asymmetric disulfide.

24. The process of claim 23, wherein said purifying is effected by column chromatography, recrystallization and/or a combination thereof.

25. The process of claim 1, wherein said allyl having a reactive group is allyl bromide, said thiol-containing compounds is captopril and said allyl-containing asymmetric disulfide is CPSSA.

26. An allyl-containing asymmetric disulfide obtained by the process of claim 1.

27. A process of preparing an allyl-containing asymmetric disulfide having the general Formula:
A-S—S—B
wherein:
A is a residue of a thiol-containing compound;
B is an allyl residue; and
A and B are different,
the process comprising reacting a compound having the general Formula B—X,
wherein X is a reactive group, with a thiol-containing compound having the general Formula A-SH, in the presence of a thiosulfate, thereby obtaining the asymmetric disulfide.

28. The process of claim 27, being a one-pot process.

29. The process of claim 27, comprising:
providing a first mixture containing said compound having the general Formula B—X and said thiosulfate;
subsequently adding a second mixture containing said compound having the general Formula A-SH to said first mixture; and
mixing said first mixture and said second mixture.

30. The process of claim 27, wherein providing said first mixture is effected by mixing said compound having the general Formula B—X and said thiosulfate for a time period that ranges from 1 hour to 20 hours.

31. The process of claim 30, wherein said time period ranges from 10 hours to 20 hours.

32. The process of claim 27, wherein said reacting is performed under basic conditions.

33. The process of claim 27, wherein said reacting is conducted at a temperature that ranges from about −50° C. to about 50° C.

34. The process of claim 27, wherein said reacting is conducted in aqueous medium.

35. The process of claim 27, wherein a molar ratio between said compound having the general Formula B—X, and said compound having the general Formula A-SH, ranges from about 10:1 to about 1:10.

36. The process of claim 35, wherein said ratio ranges from about 5:1 to about 1:5.

37. The process of claim 27, wherein a molar ratio between said compound having the general Formula B—X and said thiosulfate, ranges from about 5:1 to about 1:5.

38. The process of claim 37, wherein said ratio is about 1:1.

39. The process of claim 27, wherein X is halide.

40. The process of claim 39, wherein said halide is bromide.

41. The process of claim 27, wherein a concentration of said compound having the general Formula B—X ranges from 0.1 M to 10 M.

42. The process of claim 41, wherein said concentration ranges from about 1 M to about 5 M.

43. The process of claim 27, wherein a concentration of said thiosulfate ranges from about 0.1 M to about 10 M.

44. The process of claim 43, wherein said concentration ranges from about 1 M to about 5 M.

45. The process of claim 27, wherein said compound having the general Formula A-SH is a angiotensin-converting enzyme (ACE)-inhibiting proline derivative.

46. The process of claim 27, wherein said compound having the general Formula A-SH is captopril, a derivative or an analog thereof.

47. The process of claim 27, wherein said thiosulfate is selected from the group consisting of sodium thiosulfate, potassium thiosulfate, calcium thiosulfate, and ammonium thiosulfate.

48. The process of claim 27, further comprising purifying the allyl-containing asymmetric disulfide.

49. The process of claim 48, wherein said purifying is effected by column chromatography, recrystallization and/or a combination thereof.

50. An allyl-containing asymmetric disulfide obtained by the process of claim 27.

51. An allyl-containing asymmetric disulfide having a purity greater than 95%.

52. The allyl-containing asymmetric disulfide of claim 51, having a purity greater than 99%.

53. The allyl-containing asymmetric disulfide of claim 51, being allylmercaptocaptopril (CPSSA).

54. The process claim 27, wherein said B—X is allyl bromide, said A-SH is captopril and said allyl-containing asymmetric disulfide is CPSSA.
